(19) United States
(12) Patent Application Publication
(10) Pub. No.: US 2021/0298667 A1
(43) Pub. Date: Sep. 30, 2021

(54) System, Method, and Computer Program Product for Determining Efficacy of a Treatment Plan Using Remote Patient Monitoring

(71) Applicants: Jason Fedore, Irwin, PA (US); Craig Cox, Delmont, PA (US)

(72) Inventors: Jason Fedore, Irwin, PA (US); Craig Cox, Delmont, PA (US)

(21) Appl. No.: 17/205,233

(22) Filed: Mar. 18, 2021

Related U.S. Application Data
(60) Provisional application No. 63/000,076, filed on Mar. 26, 2020.

(51) Int. Cl. A61B 5/00

(52) U.S. Cl. CPC A61B 5/4848; A61B 5/4836; A61B 5/4824; A61B 5/0024; A61B 5/0004

(57) ABSTRACT

Provided are computer-implemented methods for determining efficacy of a treatment plan using remote patient monitoring which may include receiving data associated with operation of an electrotherapy device; receiving data associated with a treatment plan, wherein the treatment plan specifies the operation of the electrotherapy device; and determining a first value and a second value associated with an operational parameter of the electrotherapy device based on the operation of the electrotherapy device; comparing the first value to the second value. In some non-limiting embodiments or aspects, the method may include determining whether a pain level of a patient is reduced based on comparing the first value to the second value. Systems and computer program products are also provided.

### CROSS REFERENCE TO RELATED APPLICATION

This application claims the benefit of U.S. Provisional Application No. 63/000,076 filed Mar. 26, 2020, which is incorporated by reference herein in its entirety.

### BACKGROUND

### Technical Field

This disclosure relates generally to determining efficacy of a treatment plan and, in some non-limiting embodiments or aspects, to systems, methods, and computer program products for determining efficacy of a treatment plan using remote patient monitoring.

### Technical Considerations

Patients that experience physical pain (e.g., as a result of a physical injury and/or the like) may receive a treatment plan from a clinician (e.g., a doctor, a physical therapist, and/or the like). The treatment plan may specify one or more actions to be taken by the patient (e.g., to stretch, to walk for a period of time every day, to use an electrotherapy device such as a transcutaneous electrical nerve stimulation (TENS) unit, and/or the like). Additionally, the treatment plan may specify that the patient take one or more medications at one or more time intervals.

Clinicians involved in the treatment plan of the patients may have follow-up examinations or communications to determine whether the treatment plan is working (e.g., that the treatment plan corresponds to a reduction in physical pain experienced by the patient(s)). However, the clinicians may not be able to independently verify responses provided by the patients. For example, a patient may believe that they are still experiencing physical pain without a reduction in the physical pain, even though they may actually be experiencing less pain and adjusting accordingly. In such an example, the clinicians may determine whether to amend and/or stop the treatment plan based on inaccurate and/or less accurate information.

### SUMMARY

Accordingly, disclosed are systems, methods, and computer program products for determining efficacy of a treatment plan using remote patient monitoring.

According to some non-limiting embodiments or aspects, provided is a computer-implemented method for determining efficacy of a treatment plan using remote patient monitoring, the computer-implemented method including: receiving, with at least one processor, data associated with operation of an electrotherapy device; receiving, with at least one processor, data associated with a treatment plan, wherein the treatment plan specifies the operation of the electrotherapy device; determining, with at least one processor, a first value and a second value associated with an operational parameter of the electrotherapy device based on the operation of the electrotherapy device; comparing, with at least one processor, the first value to the second value; and determining, with at least one processor, whether a pain level of a patient is reduced based on comparing the first value to the second value.

According to some non-limiting embodiments or aspects, provided is a system for determining efficacy of a treatment plan using remote patient monitoring, the system including: at least one processor programmed and/or configured to: receive data associated with operation of an electrotherapy device; receive data associated with a treatment plan, wherein the treatment plan specifies the operation of the electrotherapy device; determine a first value and a second value associated with an operational parameter of the electrotherapy device based on the operation of the electrotherapy device; compare the first value to the second value; and determine whether a pain level of a patient is reduced based on comparing the first value to the second value.

According to some non-limiting embodiments or aspects, provided is a computer program product for determining efficacy of a treatment plan using remote patient monitoring, the computer program product including at least one non-transitory computer-readable medium including one or more instructions that, when executed by at least one processor, cause the at least one processor to: receive data associated with operation of an electrotherapy device; receive data associated with a treatment plan, wherein the treatment plan specifies the operation of the electrotherapy device; determine a first value and a second value associated with an operational parameter of the electrotherapy device based on the operation of the electrotherapy device; compare the first value to the second value; and determine whether a pain level of a patient is reduced based on comparing the first value to the second value.

Further non-limiting aspects or embodiments are set forth in the following numbered clauses:

Clause 1: A computer-implemented method for determining efficacy of a treatment plan using remote patient monitoring, the computer-implemented method comprising: receiving, with at least one processor, data associated with operation of an electrotherapy device; receiving, with at least one processor, data associated with a treatment plan, wherein the treatment plan specifies the operation of the electrotherapy device; determining, with at least one processor, a first value and a second value associated with an operational parameter of the electrotherapy device based on the operation of the electrotherapy device; comparing, with at least one processor, the first value to the second value; and determining, with at least one processor, whether a pain level of a patient is reduced based on comparing the first value to the second value.

Clause 2: The computer-implemented method of clause 1, further comprising: storing data associated with the first value and the second value in association with the data associated with the treatment plan in a data structure.

Clause 3: The computer-implemented method of clauses 1 or 2, wherein the data associated with the operation of the electrotherapy device comprises data associated with the operation of the electrotherapy device during a first time period, the computer-implemented method further comprising: receiving data associated with operation of the electrotherapy device during a second time period; determining a third value associated with the operational parameter of the electrotherapy device based on the operation of the electrotherapy device during the second time period; retrieving the data associated with the first value and the second value from the data structure; comparing the third value to the first value or the second value; and determining whether the pain level of the patient is reduced based on comparing the third value to the first value or the second value.

Clause 4: The computer-implemented method of any of clauses 1-3, wherein the data associated with the operation of the electrotherapy device comprises data associated with the operation of the electrotherapy device during a first time period, the computer-implemented method further comprising: receiving data associated with operation of the electrotherapy device during a second time period; determining a third value and a fourth value associated with the operational parameter of the electrotherapy device based on the operation of the electrotherapy device during the second time period; comparing the third value to the fourth value; and determining whether the pain level of the patient is reduced based on comparing the third value to the fourth value.

Clause 5: The computer-implemented method of any of clauses 1-4, further comprising: retrieving the data associated with the first value and the second value from the data structure; comparing the third value or the fourth value to the first value or the second value; and determining whether the pain level of the patient is reduced based on comparing the third value or the fourth value to the first value or the second value.

Clause 6: The computer-implemented method of any of clauses 1-5, further comprising: generating a message including an indication that the pain level of the patient is not reduced based on determining that the pain level of a patient is not reduced; and transmitting the message to an individual associated with the treatment plan.

Clause 7: The computer-implemented method of any of clauses 1-6, wherein the treatment plan specifies a medication, wherein the medication is administered at a first dose during the first time period and at a second dose during the second time period, the method further comprising: determining whether the first dose and the second dose correspond to the pain level of the patient being reduced based on determining that the pain level of the patient is reduced; generating a message including an indication of whether the first dose and the second dose correspond to the pain level of the patient being reduced; and transmitting the message to an individual associated with the treatment plan.

Clause 8: The computer-implemented method of any of clauses 1-7, further comprising: comparing operation of an electrotherapy device to the treatment plan; determining that the electrotherapy device is not being operated in accordance with the treatment plan; generating a message including an indication that the electrotherapy device is not being operated in accordance with the treatment plan; and transmitting the message to an individual associated with the treatment plan.

Clause 9: A system for determining efficacy of a treatment plan using remote patient monitoring, the system comprising: at least one processor programmed or configured to: receive data associated with operation of an electrotherapy device configured to be operated by a patient via a communication network; receive data associated with a treatment plan from a clinician device, wherein the treatment plan specifies the operation of the electrotherapy device; determine a first value and a second value associated with an operational parameter of the electrotherapy device based on the operation of the electrotherapy device; compare the first value to the second value; and determine whether a pain level of a patient is reduced based on comparing the first value to the second value.

Clause 10: A computer program product for determining efficacy of a treatment plan using remote patient monitoring, the computer program product comprising at least one non-transitory computer-readable medium comprising one or more instructions that, when executed by at least one processor, cause the at least one processor to: receive data associated with operation of an electrotherapy device configured to be operated by a patient via a communication network; receive data associated with a treatment plan from a clinician device, wherein the treatment plan specifies the operation of the electrotherapy device; determine a first value and a second value associated with an operational parameter of the electrotherapy device based on the operation of the electrotherapy device; compare the first value to the second value; and determine whether a pain level of a patient is reduced based on comparing the first value to the second value.

Clause 11: A system, comprising: at least one processor programmed or configured to perform the method of any of clauses 1-10.

Clause 12: A computer program product comprising at least one non-transitory computer-readable medium including one or more instructions that, when executed by at least one processor, cause the at least one processor to perform the method of any of clauses 1-10.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

FIG. 1 is a diagram of a non-limiting aspect or embodiment of a system for determining efficacy of a treatment plan using remote patient monitoring;

FIG. 2 is a diagram of a non-limiting aspect or embodiment of components of one or more devices and/or one or more systems of FIG. 1; and

FIG. 3 is a flowchart of a non-limiting aspect or embodiment of a process for determining efficacy of a treatment plan using remote patient monitoring.

### DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments or aspects of the disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. In addition, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and transmits the processed information to the second unit. In some non-limiting embodiments or aspects, a message may refer to a network packet (e.g., a data packet and/or the like) that includes data.

As used herein, the term “server” may refer to one or more computing devices, such as processors, storage devices, and/or similar computer components that communicate with client devices and/or other computing devices over a network, such as the Internet or private networks and, in some examples, facilitate communication among other servers and/or client devices.

As used herein, the term “system” may refer to one or more devices or combinations of devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

Provided are improved systems, methods, and computer program products for determining efficacy of a treatment plan using remote patient monitoring. In some non-limiting embodiments or aspects, systems, methods, and computer program products may include receiving, with at least one processor, data associated with operation of an electrotherapy device; receiving, with at least one processor, data associated with a treatment plan, wherein the treatment plan specifies the operation of the electrotherapy device; determining, with at least one processor, a first value and a second value associated with an operational parameter of the electrotherapy device based on the operation of the electrotherapy device; comparing, with at least one processor, the first value to the second value; and determining, with at least one processor, whether a pain level of a patient is reduced based on comparing the first value to the second value.

In this way, clinicians involved in the treatment plan of the patients may more accurately determine whether the treatment plan is working (e.g., that the treatment plan corresponds to a reduction in physical pain experienced by the patient(s)). For example, clinicians may independently verify responses provided by patients indicating whether the patients are experiencing more, the same amount, or less pain during implementation of a treatment plan. In such an example, the clinicians may verify these responses based on systems associated with the clinicians remotely monitoring operation of a user device (e.g., an electrotherapy device) to determine whether to amend and/or stop the treatment plan based on more accurate information.

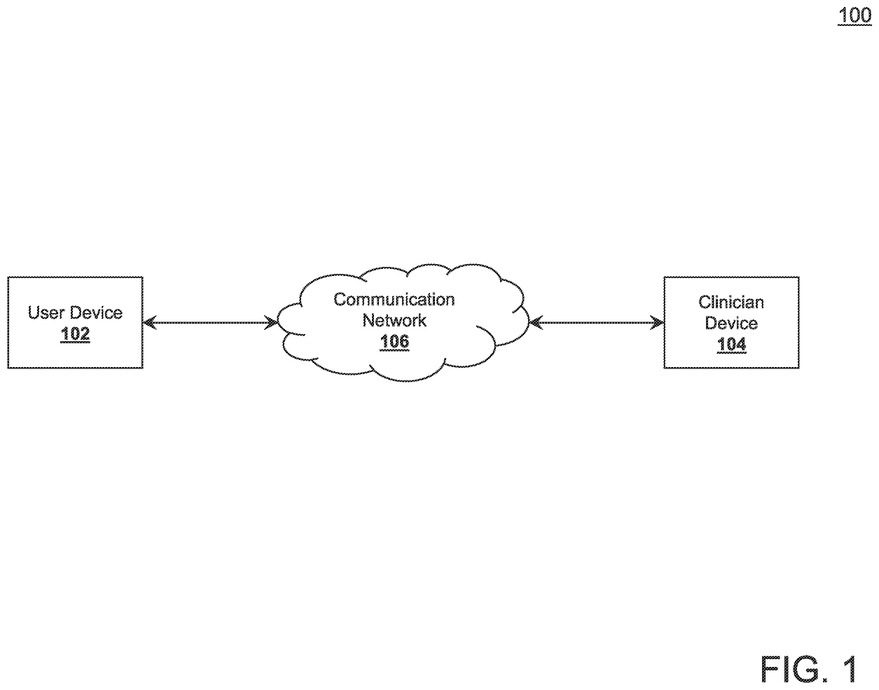

Referring now to FIG. 1, FIG. 1 is a diagram of an example environment 100 in which devices, systems, methods, and/or products described herein may be implemented. As shown in FIG. 1, environment 100 includes user device 102, clinician device 104, and communication network 106. User device 102, clinician device 104, and/or communication network 106 may interconnect (e.g., establish a connection to communicate, and/or the like) via wired connections, wireless connections, or a combination of wired and wireless connections.

User device 102 may include a device configured to be in communication with clinician device 104 via communication network 106. For example, user device 102 may include a client device, an electrotherapy device (e.g., a TENS unit, a neuromodulation devices, iontophoresis devices, ultrasonic waves, infrared therapy, electric stimulation, laser therapy, and/or light therapy devices), and/or the like. User device 102 may be configured to transmit and/or receive data to and/or from clinician device 104 via a short-range wireless communication connection (e.g., an NFC communication connection, an RFID communication connection, a Bluetooth® communication connection, and/or the like). In some non-limiting embodiments or aspects, user device 102 may be associated with a user (e.g., an individual assigned to a treatment plan). User device 102 may include one or more sensors (e.g., one or more biofeedback sensors, one or more facial and/or iris recognition sensors, one or more cameras (e.g., integral cameras and/or detachable cameras) configured to capture one or more images of a patient, one or more infrared sensors configured to measure a temperature of a patient, and/or the like) configured to receive input associated with one or more vital signs of a user. In some non-limiting embodiments or aspects, user device 102 may include one or more resistance meters configured to measure a range of motion of a portion of an anatomical feature of a patient (e.g., a coil configured to be squeezed by a hand of a patient). In some non-limiting embodiments or aspects, user device 102 may include one or more electrodes configured to be disposed about a body of a patient to measure one or more signals associated with an echocardiogram. In some non-limiting embodiments or aspects, user device 102 may include a light (e.g., an LED light) to enable two-way communication with a clinician associated with a clinician device, as described herein. In some non-limiting embodiments or aspects, user device 102 may include a spectrometer. In some non-limiting embodiments or aspects, user device 102 may include a battery (e.g., a lithium-ion battery) and/or user device 102 may be configured to charge via a universal serial bus (USB) connection and/or via a wireless charging connection (e.g., a QI charging connection). In some non-limiting embodiments or aspects, user device 102 may include a fingerprint sensor and user device 102 may be configured to measure a fingerprint and transmit data associated with the fingerprint to clinician device 104 to verify the identity of the user operating user device 102. In some non-limiting embodiments or aspects, user device 102 may include one or more processors programmed or configured to encrypt data communicated between user device 102 and clinician device 104. In some non-limiting embodiments or aspects, user device 102 may include a touch screen. In some non-limiting embodiments or aspects, user device 102 may include a device configured to receive data associated with a location of user device 102 (e.g., a global positioning system (GPS) device, and/or the like) and transmit the data associated with the location of user device 102 to clinician device 104. In some non-limiting embodiments or aspects, user device 102 may be configured to be in communication with a fitness tracking device (e.g., a pedometer, and/or the like). In some non-limiting embodiments or aspects, user device 102 may include one or more pairs of leads and/or sensors configured to be attached to a body of the user and user device 102. In some non-limiting embodiments or aspects, user device 102 may be configured to be in communication with one or more implants and/or probes of an electrotherapy device, the one or more probes configured to be positioned within a body of a patient (e.g., subcutaneously). In some non-limiting embodiments or aspects, one or more components of user device 102 may be configured to be implanted within a patient.

Clinician device 104 may include a device configured to be in communication with user device 102 via communication network 106. For example, clinician device 104 may include one or more devices such as a laptop computer, a desktop computer, a server, a group of servers, a client device, a group of client devices, and/or other like devices. In some non-limiting embodiments or aspects, clinician device 104 may be configured to communicate via a short-range wireless communication connection. In some non-limiting embodiments or aspects, clinician device 104 may be associated with a clinician (e.g., a clinician involved in a treatment plan of a user operating user device 102).

Communication network 106 may include one or more wired and/or wireless networks. For example, communication network 106 may include a cellular network (e.g., a long-term evolution (LTE) network, a third generation (3G) network, a fourth generation (4G) network, a fifth generation (5G) network, a code division multiple access (CDMA) network, and/or the like), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of some or all of these or other types of networks.

The number and arrangement of systems and/or devices shown in FIG. 1 are provided as an example. There may be additional systems and/or devices, fewer systems and/or devices, different systems and/or devices, or differently arranged systems and/or devices than those shown in FIG. 1. Furthermore, two or more systems and/or devices shown in FIG. 1 may be implemented within a single system or a single device, or a single system or a single device shown in FIG. 1 may be implemented as multiple, distributed systems or devices. Additionally, or alternatively, a set of systems or a set of devices (e.g., one or more systems, one or more devices) of environment 100 may perform one or more functions described as being performed by another set of systems or another set of devices of environment 100.

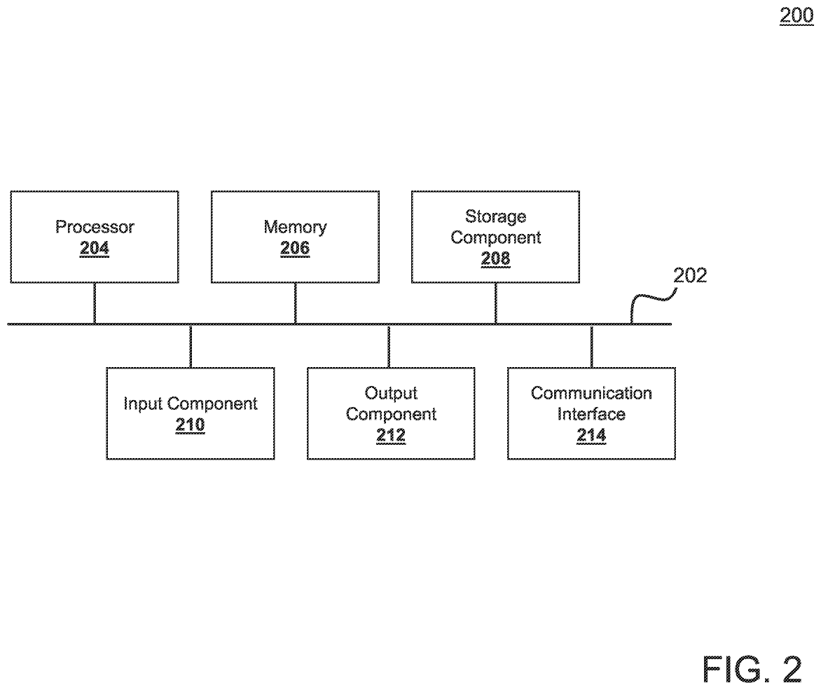

Referring now to FIG. 2, FIG. 2 is a diagram of example components of device 200. Device 200 may correspond to one or more devices of user device 102 (e.g., one or more devices of a system of user device 102), one or more devices of clinician device 104 (e.g., one or more devices of a system of clinician device 104), and/or one or more devices of the communication network 106 (e.g., one or more devices of a system of communication network 106). In some non-limiting embodiments or aspects, one or more devices of user device 102, one or more devices of clinician device 104, and/or one or more devices of the communication network 106 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like) that can be programmed to perform a function. Memory 206 may include random access memory (RAM), read-only memory (ROM), and/or another type of dynamic or static storage device (e.g., flash memory, magnetic memory, optical memory, and/or the like) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, and/or the like). Additionally or alternatively, input component 210 may include a sensor for sensing information (e.g., a global positioning system (GPS) component, an accelerometer, a gyroscope, an actuator, and/or the like). Output component 212 may include a component that provides output information from device 200 (e.g., a display, a speaker, one or more light-emitting diodes (LEDs), and/or the like).

Communication interface 214 may include a transceiver-like component (e.g., a transceiver, a separate receiver and transmitter, and/or the like) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, and/or the like). Device 200 may be capable of receiving information from, storing information in, communicating information to, or searching information stored in the data storage or one or more data structures in memory 206 and/or storage component 208. For example, the information may include input data, output data, or any combination thereof.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

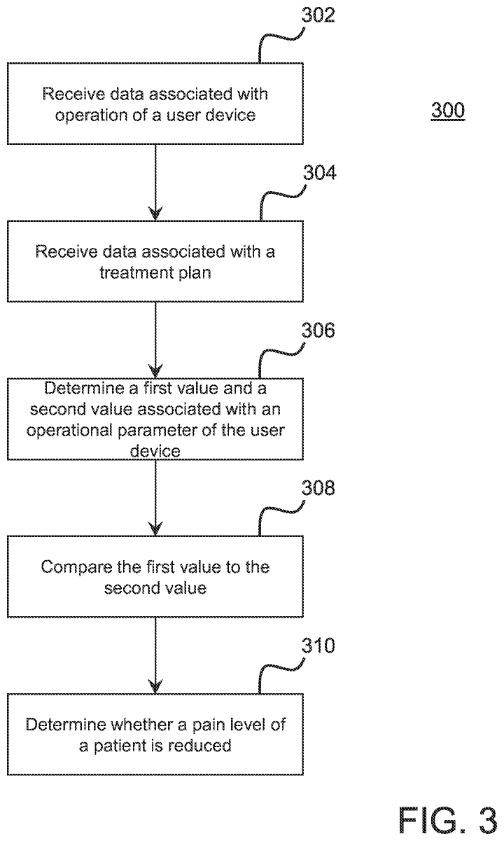

Referring now to FIG. 3, FIG. 3 is a flowchart of a non-limiting aspect or embodiment of a process 300 for determining efficacy of a treatment plan using remote patient monitoring. In some non-limiting embodiments or aspects, one or more of the functions described with respect to process 300 may be performed (e.g., completely, partially, and/or the like) by clinician device 104. In some non-limiting embodiments or aspects, one or more of the steps of process 300 may be performed (e.g., completely, partially, and/or the like) by another device or a group of devices separate from and/or including clinician device 104, such as user device 102.

As shown in FIG. 3, at step 302, process 300 may include receiving data associated with operation of a user device. For example, clinician device 104 may receive data associated with operation of user device 102. In such an example, the operation of user device 102 may be associated with a first time period. In some non-limiting embodiments or aspects, clinician device 104 may receive the data associated with the operation of user device 102 from user device 102, another clinician device, a Health Insurance Portability and Accountability Act (HIPPA) cloud-based system, and/or the like. In some non-limiting embodiments or aspects, user device 102 may receive data associated with operation of an electrotherapy device included in user device 102. In some non-limiting embodiments or aspects, the data associated with operation of user device 102 may include data associated with one or more times at which user device 102 was used by a user, one or more parameters (e.g., one or more power levels and/or the like) at which user device 102 was used by a user, data associated with one or more vital signs of the user (e.g., blood pressure, pulse, respiration rate, temperature, and/or the like), and/or the like. In some non-limiting embodiments or aspects, clinician device 104 may receive data associated with operation of user device 102 based on (e.g., before, during, after, in response to, and/or the like) operation of user device 102. For example, clinician device 104 may receive data associated with operation of user device 102 based on operation of user device 102 by a user associated with user device 102 in accordance with a treatment plan, as described herein.

As shown in FIG. 3, at step 304, process 300 may include receiving data associated with a treatment plan. For example, clinician device 104 may receive data associated with a treatment plan. The treatment plan may involve a user associated with user device 102 and a clinician associated with clinician device 104. In such an example, clinician device 104 may receive data associated with a treatment plan based on input provided to clinician device 104 and/or from one or more other devices (e.g., another clinician device 104 associated with another clinician involved in the treatment plan and/or the like). In some non-limiting embodiments or aspects, the treatment plan may specify operation of user device 102 and/or administration of one or more medications (e.g., analgesics, anti-inflammatory drugs, and/or the like), alone or in combination. For example, the treatment plan may specify administration of one or more medications at one or more doses at one or more times (e.g., one or more times during a first time period). In an example, the treatment plan may specify administration of a medication at a first dose during a first time and administration of the medication at a second dose during a second time. The treatment plan may also specify use of user device 102 during the first time period, the second time period, and/or a time period different from the first time period or the second time period. In some non-limiting embodiments or aspects, clinician device 104 may transmit data associated with the treatment plan. For example, clinician device 104 may transmit data associated with the treatment plan to user device 102. In such an example, the data associated with the treatment plan may be configured to cause user device 102 to display an image associated with the treatment plan (e.g., one or more images that provide information about the treatment plan).

As shown in FIG. 3, at step 306, process 300 may include determining a first value and a second value associated with an operational parameter of a user device. For example, clinician device 104 may determine a first value associated with (e.g., representing) an operational parameter of user device 102 and clinician device 104 may determine a second value associated with (e.g., representing) the operational parameter of user device 102. In some non-limiting embodiments or aspects, an operational parameter may include a level of intensity of electronic stimulation provided by user device 102 to the user associated with user device 102, a frequency of electronic stimulation provided by user device 102 to the user associated with user device 102, a duration of electronic stimulation provided by user device 102 to the user associated with user device 102, a time (e.g., a time, a time and date, and/or the like) of electronic stimulation provided by user device 102 to the user associated with user device 102, and/or the like. In some non-limiting embodiments or aspects, the first value may be associated with a first time and the second value may be associated with a second time. In some non-limiting embodiments or aspects, the first time may include a first range of times and/or the second time may include a second range of times.

In some non-limiting embodiments or aspects, clinician device 104 may determine the first value and the second value associated with the operational parameter of user device 102 based on operation of user device 102. Additionally or alternatively, clinician device 104 may determine the first value and/or the second value associated with the operational parameter of user device 102 based on clinician device 104 determining that the user associated with user device 102 forewent using user device 102. For example, clinician device 104 may determine that the treatment plan specified use of user device 102 at one or more times and clinician device 104 may determine that the user associated with user device 102 did not use user device 102 at the one or more times. In such an example, clinician device 104 may determine that the first value and/or the second value are associated with a predetermined value, the predetermined value associated with non-use of user device 102 at a time.

In some non-limiting embodiments or aspects, clinician device 104 may store data associated with the first value and/or the second value in association with data associated with the treatment plan. For example, clinician device 104 may store data associated with the first value and/or the second value in association with data associated with the treatment plan in a data structure. In such an example, the data structure may be configured to be in communication with user device 102, clinician device 104, and/or one or more other clinician devices 104.

As shown in FIG. 3, at step 308, process 300 may include comparing the first value to the second value. For example, clinician device 104 may compare the first value to the second value. In such an example, clinician device 104 may compare the first value to the second value based on clinician device 104 determining the first value and the second value. In some non-limiting embodiments or aspects, clinician device 104 may determine that the first value is greater than, equal to, and/or less than the second value based on clinician device 104 comparing the first value and the second value. For example, clinician device 104 may determine that the first value is greater than, equal to, and/or less than the second value and clinician device 104 may determine whether the treatment plan is effective (e.g., that the user associated with user device 102 is experiencing less pain based on actions taken in accordance with the treatment plan) or ineffective (e.g., that the user associated with user device 102 is experiencing the same amount of pain and/or a greater amount of pain based on actions taken in accordance with the treatment plan). In such an example, clinician device 104 may determine that the treatment plan is effective based on the first value being greater than the second value (e.g., indicating that user device 102 was used at a higher intensity at the first time than at the second time). Additionally or alternatively, clinician device 104 may determine that the treatment plan is ineffective based on the first value being less than the second value (e.g., indicating that user device 102 was used at a higher intensity at the second time than at the first time).

As shown in FIG. 3, at step 310, process 300 may include determining whether a pain level of a patient is reduced. For example, clinician device 104 may determine whether a pain level of a patient is reduced. In some non-limiting embodiments or aspects, clinician device 104 may determine whether the pain level of the patient is reduced based on operation of user device 102. For example, clinician device 104 may determine whether the pain level of the patient is reduced based on the first value and the second value. In such an example, clinician device 104 may compare the first value to the second value to determine whether the treatment plan is effective or ineffective and clinician device 104 may determine whether the pain level of the patient is reduced based on clinician device 104 determining whether the treatment plan is effective or ineffective. In some non-limiting embodiments or aspects, clinician device 104 may determine that the pain level of the patient is reduced based on clinician device 104 determining that the treatment plan is effective. Additionally or alternatively, clinician device 104 may determine that the pain level of the patient is not reduced based on clinician device 104 determining that the treatment plan is ineffective.

In some non-limiting embodiments or aspects, clinician device 104 may generate a message configured to provide an indication of whether the pain level of the patient is reduced or not reduced. For example, clinician device 104 may generate a message configured to provide an indication of whether the pain level of the user associated with user device 102 is reduced or not reduced based on clinician device 104 determining whether the pain level of the user is reduced. In such an example, the message may include data associated with output (e.g., an image) that indicates whether the pain level of the user is reduced or not reduced.

In some non-limiting embodiments or aspects, clinician device 104 may provide the message configured to provide an indication of whether the pain level of the patient is reduced or not reduced to one or more components of clinician device 104. For example, clinician device 104 may provide (e.g., transmit) the message configured to provide an indication of whether the pain level of the patient is reduced or not reduced to a display device of clinician device 104. In such an example, the display device of clinician device 104 may generate and provide the indication (e.g., an image) as output indicating whether the pain level of the patient is reduced or not reduced.

In some non-limiting embodiments or aspects, clinician device 104 may determine one or more pain levels corresponding to one or more time periods during which user device 102 was used by a user. For example, clinician device 104 may determine one or more pain levels corresponding to one or more time periods during which user device 102 was used by a user, where the one or more pain levels are represented as pain levels measured on a pain level scale (e.g., a numeric rating scale (NRS), a visual analog scale (VAS), a categorical pain scale (e.g., a scale from 1-10, a scale represented by one or more images such as illustrated smiley faces at different pain levels, and/or the like), and/or the like). In some non-limiting embodiments or aspects, clinician device 104 may receive input from a clinician to adjust a treatment plan. For example, clinician device 104 may receive input from a clinician to adjust a treatment plan based on (e.g., after) displaying an indication of whether the pain level of the user is reduced. In such an example, clinician device 104 may transmit data associated with the treatment plan that was updated to the data structure including the data associated with the treatment plan. In some non-limiting embodiments or aspects, updating the treatment plan may include updating a treatment method, updating a time duration during which user device 102 is to be used by the user associated with user device 102, updating instructions associated with placement of one or more components of user device 102 (e.g., electrode pads) relative to the user associated with user device 102 during use of user device 102, and/or the like.

In some non-limiting embodiments or aspects, clinician device 104 may receive data associated with operation of user device 102 during a second time period (e.g., a time period after the first time period). The data associated with operation of user device 102 during the second time period may include data associated with one or more times at which user device 102 was used by the user associated with user device 102, one or more parameters at which user device 102 was used by the user associated with user device 102, and/or the like. In some non-limiting embodiments or aspects, clinician device 104 may receive data associated with operation of user device 102 based on (e.g., before, during, after, in response to, and/or the like) operation of user device 102 during the second time period.

In some non-limiting embodiments or aspects, clinician device 104 may determine a third value and/or a fourth value associated with the operational parameter of user device 102 in a similar manner to clinician device 104 determining the first value and the second value associated with the operational parameter of user device 102. For example, clinician device 104 may determine the third value and/or the fourth value associated with the operational parameter of user device 102 based on operation of user device 102 during the second time period. Additionally or alternatively, clinician device 104 may determine the third value and/or the fourth value associated with the operational parameter of user device 102 based on clinician device 104 determining that the user associated with user device 102 forewent using user device 102 during a portion of and/or all of the second time period. For example, clinician device 104 may determine that the treatment plan specified use of user device 102 at one or more times during the second time period and clinician device 104 may determine that the user associated with user device 102 did not use user device 102 at the one or more times during the second time period. In such an example, clinician device 104 may determine that the third value and/or the fourth value are associated with the predetermined value associated with non-use of user device 102.

In some non-limiting embodiments or aspects, clinician device 104 may store data associated with the third value and/or the fourth value in association with data associated with the treatment plan, the first value, and/or the second value. For example, clinician device 104 may store data associated with the third value and/or the fourth value in association with data associated with the treatment plan, the first value, and/or the second value in the data structure described herein.

In some non-limiting embodiments or aspects, clinician device 104 may retrieve the data associated with the first value and the second value from the data structure. For example, clinician device 104 may retrieve the data associated with the first value and the second value from the data structure based on clinician device 104 receiving data associated with operation of user device 102 during the second time period. Additionally or alternatively, clinician device 104 may retrieve the data associated with the first value and the second value from the data structure based on clinician device 104 determining the third value and/or the fourth value, as described herein.

In some non-limiting embodiments or aspects, clinician device 104 may compare the third value to the fourth value. For example, clinician device 104 may compare the third value to the fourth value based on clinician device 104 determining the third value and the fourth value. In some non-limiting embodiments or aspects, clinician device 104 may determine that the third value is greater than, equal to, and/or less than the fourth value. For example, clinician device 104 may determine that the third value is greater than, equal to, and/or less than the fourth value and clinician device 104 may determine whether the treatment plan is effective (e.g., that the user associated with user device 102 is experiencing less pain based on actions taken in accordance with the treatment plan) or ineffective (e.g., that the user associated with user device 102 is experiencing the same amount of pain and/or a greater amount of pain based on actions taken in accordance with the treatment plan). In such an example, clinician device 104 may determine that the treatment plan is effective based on the third value being greater than the fourth value (e.g., indicating that user device 102 was used at a higher intensity at the third time than at the fourth time). Additionally or alternatively, clinician device 104 may determine that the treatment plan is ineffective based on the third value being less than the fourth value (e.g., indicating that user device 102 was used at a higher intensity at the fourth time than at the third time).

In some non-limiting embodiments or aspects, clinician device 104 may compare the third value and/or the fourth value to the first value and/or the second value. For example, clinician device 104 may compare the third value and/or the fourth value to the first value and/or the second value based on clinician device 104 determining the third value and/or the fourth value. In some non-limiting embodiments or aspects, clinician device 104 may determine that the third value and/or the fourth value is/are greater than, equal to, and/or less than the first value and/or the second value. For example, clinician device 104 may determine that the third value and/or the fourth value is/are greater than, equal to, and/or less than the first value and/or the second value and clinician device 104 may determine whether the treatment plan is effective (e.g., that the user associated with user device 102 is experiencing less pain based on actions taken in accordance with the treatment plan) or ineffective (e.g., that the user associated with user device 102 is experiencing the same amount of pain and/or a greater amount of pain based on actions taken in accordance with the treatment plan). In such an example, clinician device 104 may determine that the treatment plan is effective based on the first value and/or the second value being greater than the third value and/or the fourth value (e.g., indicating that user device 102 was used at a higher intensity during the first time period (e.g., at the first time and/or the second time) than during the second time period (e.g., at the third time and/or a fourth time)). Additionally or alternatively, clinician device 104 may determine that the treatment plan is ineffective based on the first value and/or the second value being less than the third value and/or the fourth value (e.g., indicating that user device 102 was used at a higher intensity during the second time period (e.g., at the third time and/or the fourth time) than during the first time period (e.g., at the first time and/or the second time)).

In some non-limiting embodiments or aspects, clinician device 104 may determine whether the pain level of the patient is reduced based on operation of user device 102 during the first time period and/or the second time period. For example, clinician device 104 may determine whether the pain level of the patient is reduced based the third value and/or the fourth value obtained during the second time period. In such an example, clinician device 104 may compare the third value to the fourth value to determine whether the treatment plan is effective or ineffective. Additionally or alternatively, clinician device 104 may compare the third value and/or the fourth value to the first value and/or the second value to determine whether the treatment plan is effective or ineffective.

Although the above systems, methods, and computer program products have been described in detail for the purpose of illustration based on what is currently considered to be the most practical and preferred embodiments or aspects, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A computer-implemented method for determining efficacy of a treatment plan using remote patient monitoring, the computer-implemented method comprising:
receiving, with at least one processor, data associated with operation of an electrotherapy device;
receiving, with at least one processor, data associated with a treatment plan, wherein the treatment plan specifies the operation of the electrotherapy device;
determining, with at least one processor, a first value and a second value associated with an operational parameter of the electrotherapy device based on the operation of the electrotherapy device;
comparing, with at least one processor, the first value to the second value; and
determining, with at least one processor, whether a pain level of a patient is reduced based on comparing the first value to the second value.

2. The computer-implemented method of claim 1, further comprising:
storing data associated with the first value and the second value in association with the data associated with the treatment plan in a data structure.

3. The computer-implemented method of claim 2, wherein the data associated with the operation of the electrotherapy device comprises data associated with the operation of the electrotherapy device during a first time period, the computer-implemented method further comprising:
receiving data associated with operation of the electrotherapy device during a second time period;
determining a third value associated with the operational parameter of the electrotherapy device based on the operation of the electrotherapy device during the second time period;
retrieving the data associated with the first value and the second value from the data structure;
comparing the third value to the first value or the second value; and
determining whether the pain level of the patient is reduced based on comparing the third value to the first value or the second value.

4. The computer-implemented method of claim 2, wherein the data associated with the operation of the electrotherapy device comprises data associated with the operation of the electrotherapy device during a first time period, the computer-implemented method further comprising:
receiving data associated with operation of the electrotherapy device during a second time period;
determining a third value and a fourth value associated with the operational parameter of the electrotherapy device based on the operation of the electrotherapy device during the second time period;
comparing the third value to the fourth value; and
determining whether the pain level of the patient is reduced based on comparing the third value to the fourth value.

5. The computer-implemented method of claim 4, further comprising:
retrieving the data associated with the first value and the second value from the data structure;
comparing the third value or the fourth value to the first value or the second value; and
determining whether the pain level of the patient is reduced based on comparing the third value or the fourth value to the first value or the second value.

6. The computer-implemented method of claim 1, further comprising:
generating a message including an indication that the pain level of the patient is not reduced based on determining that the pain level of a patient is not reduced; and
transmitting the message to an individual associated with the treatment plan.

7. The computer-implemented method of claim 4, wherein the treatment plan specifies a medication, wherein the medication is administered at a first dose during the first time period and at a second dose during the second time period, the method further comprising:
determining whether the first dose and the second dose correspond to the pain level of the patient being reduced based on determining that the pain level of the patient is reduced;
generating a message including an indication of whether the first dose and the second dose correspond to the pain level of the patient being reduced; and
transmitting the message to an individual associated with the treatment plan.

8. The computer-implemented method of claim 1, further comprising:
comparing operation of an electrotherapy device to the treatment plan;
determining that the electrotherapy device is not being operated in accordance with the treatment plan;
generating a message including an indication that the electrotherapy device is not being operated in accordance with the treatment plan; and
transmitting the message to an individual associated with the treatment plan.

9. A system for determining efficacy of a treatment plan using remote patient monitoring, the system comprising:
at least one processor programmed or configured to:
receive data associated with operation of an electrotherapy device configured to be operated by a patient via a communication network;
receive data associated with a treatment plan from a clinician device, wherein the treatment plan specifies the operation of the electrotherapy device;
determine a first value and a second value associated with an operational parameter of the electrotherapy device based on the operation of the electrotherapy device;
compare the first value to the second value; and
determine whether a pain level of a patient is reduced based on comparing the first value to the second value.

10. A computer program product for determining efficacy of a treatment plan using remote patient monitoring, the computer program product comprising at least one non-transitory computer-readable medium comprising one or more instructions that, when executed by at least one processor, cause the at least one processor to:
receive data associated with operation of an electrotherapy device configured to be operated by a patient via a communication network;
receive data associated with a treatment plan from a clinician device, wherein the treatment plan specifies the operation of the electrotherapy device;
determine a first value and a second value associated with an operational parameter of the electrotherapy device based on the operation of the electrotherapy device;
compare the first value to the second value; and
determine whether a pain level of a patient is reduced based on comparing the first value to the second value.

11. A system, comprising:
at least one processor programmed or configured to perform the method of claim 1.

12. A computer program product comprising at least one non-transitory computer-readable medium including one or more instructions that, when executed by at least one processor, cause the at least one processor to perform the method of claim 1.
